(19) United States
(12) Patent Application Publication
(10) Pub. No.: US 2011/0281321 A1
(43) Pub. Date: Nov. 17, 2011

(54) Microbial Remediation System and Method

(76) Inventor: Paul SKILLICORN, Austin, TX (US)

(21) Appl. No.: 13/105,486

(22) Filed: May 11, 2011

Related U.S. Application Data
(60) Provisional application No. 61/333,928, filed on May 12, 2010.

(51) Int. Cl. C12N 11/12; A62D 3/02

(52) U.S. Cl. 435/179; 435/262.5

(57) ABSTRACT

Methods and systems for effecting an environmental change by providing an effector composition that includes lignocellulosic particles with at least one absorbed microorganism species, wherein the lignocellulosic particles are preferably kenaf particles. The method steps include mixing at least one microorganism species with kenaf particles to form an effector composition and then dispersing the effector composition in the environment.

### CROSS-REFERENCE TO RELATED APPLICATIONS

The present invention is related to one or more prior-filed co-pending patent applications and claims priority therefrom; it claims the benefit from U.S. Provisional Patent Application Ser. No. 61/333,928 filed May 12, 2010, which is incorporated herein by reference in its entirety.

### BACKGROUND OF THE INVENTION

1. Field of the Invention

The present invention relates to bioremediation.

2. Description of the Prior Art

Prior art includes US Patent Application 20020169080 for a composition for oil and gas drilling fluids with solidification agent, cell transport agent and cellulosic additive. The composition for oil and gas drilling fluids containing organic contaminants includes a solidification agent; cell transport agent; a cellulosic additive; and wherein said composition comprises enough solidification agent to enable the solidification agent to electrolytically attract the organic contaminates to the surface of the solidification agent when hydrated.

### SUMMARY OF THE INVENTION

The present invention relates to a composition and method for effecting environmental change with microorganisms absorbed onto a lignocellulosic particle.

It is an object of this invention to provide an effector composition that includes lignocellulosic particles, most preferably kenaf particles, with at least one absorbed microorganism species for the purpose of effecting an environmental change.

Another object of this invention is to provide a method for effecting an environmental change by providing an effector composition that includes kenaf particles with at least one absorbed microorganism species.

Accordingly, a broad embodiment of this invention is directed to kenaf particles loaded with bacteria for the purpose of degrading toxins in the environment.

These and other aspects of the present invention will become apparent to those skilled in the art after a reading of the following description of the preferred embodiment when considered with the drawings, as they support the claimed invention.

### DETAILED DESCRIPTION

Referring now to the drawings in general, the illustrations are for the purpose of describing a preferred embodiment of the invention and are not intended to limit the invention thereto. U.S. Pat. No. 7,481,934 and US Application Publication No. 20090170747, US Application Publication No. 20080206836, each of which are incorporated herein by reference in their entirety.

The present invention provides an effector composition that includes a lignocellulosic particle with at least one absorbed microorganism species. In the present invention, an effector composition is an entity having the ability to effect a change on a target environment.

In methods according to the present invention, methods for effecting an environmental bioremediation include the steps of: providing an effector composition including lignocellulosic particles with at least one absorbed microorganism species therein, and more particularly, providing an effector composition including lignocellulosic particles; and mixing at least one microorganism species with the lignocellulosic particles to form an effector composition. A further step of absorbing the at least one microorganism species into internal spaces of the lignocellulosic particles may be included in the methods of the present invention in addition to the foregoing steps. Another further step of dispersing the effector composition in a target environment for effecting bioremediation thereof may be included in the methods of the present invention in addition to the foregoing steps. In the foregoing methods, preferably, the lignocellulosic particles include kenaf core particles, and more preferably, the lignocellulosic particles consist substantially of kenaf particles.

The present invention also provides systems for effecting an environmental bioremediation including an effector composition including lignocellulosic particles with at least one absorbed microorganism species therein. In the foregoing methods, preferably, the lignocellulosic particles include kenaf core particles, and more preferably, the lignocellulosic particles consist substantially of kenaf particles.

Importantly, lignocellulosic particle provides both structure and media to the bacteria, facilitating the delivery and growth of the bacteria at the target site. Additionally, the particle can absorb environmental contaminants.

The lignocellulosic particle is preferably a powdered natural lignocellulosic material and can consist of sphagnum moss, hemp hurd, jute stick, balsa wood, other hard and soft woods, kenaf core, crop straws, grass specie stems, bamboo specie stems, reed stalks, peanut shells, coconut husks, pecan shells, other shells, rice husk, other grain husks, corn stover, other grain stalks, cotton stalk, sugar cane bagasse, conifer and hardwood barks, corn cobs, and combinations thereof.

As set forth hereinabove, preferably, the particle is powdered kenaf core. Beneficially, the kenaf core has a porous structure and large specific surface area which will accommodate more microorganisms, including bacterial, algal and/or fungal agents, than any other known natural material. Bacterial, algal and/or fungal agents will attach to kenaf core whether in aqueous, moist or completely dry circumstances. Thus, a wide range of bacterial, algal and/or fungal agents can be accommodated in, on and within small-to-medium-sized particles of kenaf core and delivered by that mechanism to select targeted application.

The 100% natural kenaf core eventually degrades completely, leaving no trace—consumed, in part, by the microbes it naturally carries. The objective of “delivering a result while also disappearing completely” is served.

Powdered kenaf core has a large specific surface area, approximately 100-500 square meters per gram. This large specific surface area serves multiple purposes. It allows for the absorption of a large amount of microorganism per gram of powder. It also allows for the absorption of a large amount of environmental toxin or other target molecule. The high surface area of the kenaf core enhances the probability of interaction between the “loaded” microbes through absorption/adsorption/flocced aggregation of the targeted material. Pharmaceutical residuals, toxic agents such as PCBs, aromatic compounds, and petroleum-derived materials can all be “picked up efficiently” by kenaf core. These two effects, absorption of microorganisms and environmental molecules, synergize to produce a rapid and sustained interaction between the microorganism and the target molecule. Thus, the present invention is faster and more efficient than prior art applications at bioremediation.

The slow, continuing breakdown of the kenaf core material guarantees ongoing delivery of microbes for an extended period of time and it also continues to provide them with sustenance by delivering sugar to the microbes as the kenaf cellulose and hemicellulose substrate are hydrolyzed. This enhanced carbon/nitrogen ratio can, at the margin, help to ensure a much more vigorous interaction between the microbes and the target substance.

The kenaf can be used alone or in combination with other particulates, including, for example, sphagnum moss. Alternatively, sphagnum moss can be used without kenaf.

In yet another preferred embodiment, the microorganism species include at least one species that produces a cellulase. The cellulase increases the breakdown and release of the glucose from the cellulose and hemicelluloses. Note that there are also available microbes that produce hemicellulase, laccase, pectinase and other relevant enzymes.

The microorganisms' slow, continuous breakdown of the kenaf core material is guaranteed to release everything at or proximate to the targeted application over an extended period of time—delivery timing being a function of particle size.

Thus, the present invention provides a method for effecting an environmental change on a target environment by providing an effector composition that includes lignocellulosic particles with at least one absorbed microorganism species. The lignocellulosic particles can be kenaf particles. The method steps include mixing the microorganism with the kenaf particle to form an effector composition and then dispersing the effector composition in the environment.

Target applications include any of the applications wherein a microorganism is used to effect a change. Such applications include single microorganism species and combinations of microorganism species to treat liquids, solids, surfaces and other environments. Some examples include, but are not limited to, biofertility, biopesticides, bio-yield enhancements, cleaning and odor control, direct-fed microbials, waste degradation, aquaculture waste degradation, bioaugmentation and remediation, surface cleanup and remediation, wastewater treatment, drain line and septic maintenance, pet and aquarium care, carpet cleaning, and the like.

Additionally, effector compositions can be those which secrete enzymes and other molecules that can digest or otherwise degrade molecules such as long-chain fatty acids, proteins, lipids, carbohydrates; fats, oils and greases; phenols, creosols and related aromatic compounds, petroleum and petroleum fractions and other hydrocarbons, nonionic and anionic surfactants and their components, including aromatics and fatty acids, ketones, recalcitrant organics. Effector compositions that function at various temperatures, including at low and high temperatures, and in various atmosphere, including anaerobic, aerobic, or facultative, are also within the scope of the present invention.

Establishing and enhancing nitrification is yet another application of the present invention. In each of the following examples, the present invention provides for systems and/or methods for effecting an environmental change on a target environment and/or object by providing an effector composition that includes lignocellulosic particles with at least one absorbed microorganism species therewith and/or therewithin (absorbed and/or positioned in space between the particles) lignocellulosic particles. It is preferably kenaf core particles and/or powder that are loaded with the at least one microorganism species.

Oils fall within a special category for absorption/adsorption, notably in circumstances where they are in contact with (floating on) water. These oils can include everything from light consumables such as rapeseed oil to heavy crude and oil-saturated drilling sludges.

EXAMPLE: The following provides an example for an oil spill on water as the target environment for bioremediation. Lignocellulosic particles, preferably kenaf core particles and/or powder are loaded with hydrocarbon-degrading agents, such as bacteria, algae and/or fungi, and then delivered by a wide variety of means and in a wide variety of forms to the surface of the water body experiencing the spill. Since kenaf can absorb/adsorb oil up to 11 times its weight and do so in absolute preference to water, the immediate effect is to achieve a dramatic aggregation of the oil. The physical structure thus imparted to the now aggregated oil allows it to be picked or scooped up with relative ease. This material may then be taken to a disposal site and simply left to degrade—with the microbial agents now able efficiently to do the work intended of them. In circumstances where the affected area is too large, or there is a shortage of vessels (or means) by which to pick up the aggregated oil, simple superficial delivery of the microbe-loaded kenaf will, nevertheless, serve to get the job done. Oil is first aggregated into clumps, thus rendering it immediately less harmful to those birds, fish and other animals that frequent the water surface. Those clumps also serve to bring the oil into sustained contact with the microbes that have been loaded into/onto the kenaf—allowing them to immediately start breaking down the oil.

EXAMPLE: The following provides an example for an oil spill on land and/or water making landfall as the target environment for bioremediation. Lignocellulosic particles, preferably kenaf core particles and/or powder are loaded with hydrocarbon-degrading agents and then delivered to the surface of the affected area—preferably worked, gently, into the surface and then irrigated occasionally with warm water. The water will have the effect of helping to bring the oil to the surface where the kenaf will then capture and hold it, bringing the microbes that have been loaded into/onto the kenaf into close and sustained contact with the oil and allowing them efficiently to degrade the oil. Left as such, the oil and the kenaf will simply disappear and the soil/sand will have been remediated.

EXAMPLE: The following provides an example for oil on feathers and/or fur as the target “environment” or target object(s) for bioremediation. Lignocellulosic particles, preferably kenaf core particles and/or powder are loaded with hydrocarbon-degrading agents and then fluffed/worked into the feathers or fur of the affected animal. The oil is picked up by the kenaf core which can then easily be “fluffed out” of the feathers or brushed out of the fur. The embedded microbes meanwhile continue to break down the oil. This approach is much gentler than use of harmful solvents. It is also more effective at both removing the oil and subsequently degrading it.

EXAMPLE: The following provides an example for activated sludge wastewater treatment as the target environment for bioremediation. Every activated sludge wastewater treatment plant occasionally experiences a circumstance wherein their “mixed liquor” is wiped out, degraded, washed away or simply rendered “unbalanced.” Bringing it back involves adding bacterial agents—notably those bacteria responsible for breaking down ammonia into nitrate/nitrite and those subsequently responsible for releasing the nitrogen in nitrate as harmless nitrogen gas. Loading these bacteria into/onto powdered kenaf core is a particularly effective means by which to bring a wastewater treatment plant “back” following such incidents. While the kenaf is an effective and efficient means by which to deliver the bacteria, it also serves as an efficient, ongoing platform for these organisms to continue doing their work. Continued application of “plain” kenaf core can help to maintain this higher level of efficiency indefinitely, into the future.

EXAMPLE: The following provides an example for aerobic digestion of waste material as the target environment for bioremediation. Removal/disposal of sewage sludge is now reputed to account for 60 cents of every dollar spent on wastewater treatment. Reducing the physical volume of the material requiring disposal has now begun to preoccupy managers of treatment facilities. Supplementation with powdered kenaf core has already been demonstrated to improve, significantly, the reduction in final solids disposal in both anaerobic and aerobic digestion of waste activated sludge solids. The kenaf has been shown to improve the carbon/nitrogen ratio, enhance interaction among bacteria and mitigate the inhibitor effects of ECP (extra-cellular polysaccarides) material. Additional supplementation with microbe-loaded kenaf can also help better manage the successor bacteria sequence by which sludge is broken down and thus improve the speed at which sludge is eliminated.

EXAMPLE: The following provides an example for composting as the target environment for bioremediation. Composting of kitchen waste is practiced by millions of households across the world. With few exceptions, these composting operations suffer from three “problems:” (a) there is too much nitrogen relative to available carbon, and (b) the “pile” or “bin” suffers from a shortage of air/oxygen (not turned enough), and (c) the operation needs a “jump start.” Addition of kenaf helps solves the first two problems. The bacteria delivered by a bacterially-loaded kenaf will solve the third.

EXAMPLE: The following provides an example anaerobic digestion of waste material as the target environment for bioremediation. Kenaf provides the same benefits in this circumstance as it does to aerobic digestion: “get started” with right organisms, improve C/N ratios, mitigate ECP effects. Results will deliver more methane and reduce final solids to be disposed of.

EXAMPLE: The following provides an example for timed delivery of digestive aids and beneficial fiber to the human gut as the target environment for bioremediation. The gut is all about bacteria working on what humans consume and then subsequently delivering the nutrients liberated by the process through the gut-wall into the blood stream. The food humans eat today is deficient in fiber and our guts have become colonized with “suboptimal bacterial mixes.” Kenaf powder selectively loaded with targeted bacteria can help overcome this problem—delivering fiber and helping, simultaneously, to improve the bacterial mix in our guts. The kenaf delivery mechanism can also help bypass the stomach and deliver the bacteria more quickly to the gut where they must eventually do their work.

EXAMPLE: The following provides an example for potting soil supplements as the target environment for bioremediation Potting soil supplements—enhancing the subsoil bacterial mix while also improving subsoil aeration and “movement” of organisms. Improvements here, in every respect can be profound—with enhancements provided by the kenaf's chemical and physical makeup as well as by the microbes that have been “loaded” onto/into the kenaf.

In some testing, the following mixing were used and compared: 0.015 gallons of microorganism bioremediation solution per pound of kenaf using a simple misted aerosol airstream combination process and then applied the Activated Kenaf (effector composition or substance) at the rate 0.42 g of Activated Kenaf to 0.5 grams of oil and obtained almost exactly the same efficacy results. This was “loaded” onto kenaf very effectively and maintained its viability for a significant amount of time; however, it was preferred to be using the mixed material within the next few days after mixing. The results made it clear that the kenaf was acting as a bioreactor and a source of nutrition for the bacteria, adding that the fact that it absorbed oil so effectively that it greatly favored bring the bacteria and the oil together under the most favorable circumstance. The nutrition provided by the kenaf is the sugar released as the kenaf gradually breaks down.

In methods of the present invention, the application of the effector composition or substance, “loaded kenaf” essentially “infect” all of the “contiguous amounts (or bits) of oil in the targeted environment for remediation. Surprisingly, the results proved that at less than one tenth the original concentration of bacteria (without combination to lignocellulosic material like kenaf) used in the same formal testing procedure, producing equally good results for bioremediation.

These methods in another example were combined for a swamp-sea type environment and include the following:

1. Finely powdered kenaf loaded with bacteria applied by air at rates between 1,000 and 4,000 lbs per acre. 2. Coarsely powdered kenaf loaded with bacteria and then fully saturated with water and applied hydraulically (poured or sprayed) to targeted areas thought to have higher “on the bottom” concentrations of oil. 3. Coarsely powdered kenaf loaded with bacteria and then enclosed in inch-thick mats and 4″ diameter booms, which are subsequently fully saturated with water and then anchored to the bottom in “channels” where oil is known to be “moving back and forth within the swamp” as tides go in and out. Thus, the application goal, statistically, approaches 100% as to the probability of infecting every “piece or amount” of oil in the marshes.

The following table, TABLE 1, provides test data supporting the effectiveness and efficiency of bioremediation of a target environment using the effector composition of the present invention for bioremediation of crude oil in the ocean. The test protocol included evaluation of the biodegradation of crude oil in sea water in a worst-case scenario. The test measured how well the effector composition reduced total Alkanes and total aromatics compared to a control group. The effector composition according to the present invention included the mixing of at least one microorganism species substance in the amount of about one (1) gallon to 75 pounds of kenaf material particles. The effector composition was added to flasks containing crude oil and clean seawater. Before adding the seawater, oil was prepared by being heated to 521 degrees Fahrenheit to remove lighter hydrocarbons and kill all present microorganisms. The flasks were sealed and shaken, but not aerated, until removed for sampling on 7 days and 28 days. The results are shown in the tables below, namely TABLE 1 Summary Data Table, and TABLE 2 Product comparison Chart, and showed 85.2% reduction of total Alkanes and 11% of the aromatics in 7 days; and 99.8% reduction of Alkanes and 71% reduction of aromatics in 28 days, thereby meeting the criteria stipulated by US EPA for a bioremediation agent, according to the EPA National Contingency Plan Product Schedule effective at the time of the present invention, which is incorporated herein by reference for that version in its entirety.

TABLE 1
Summary Data Table

| | | Total Mean | | Total Mean | |
|---|---|---|---|---|---|
| | Product | Alkanes | Reduction | Aromatics | Reduction |
| Days | 3 Reps/Prod | (ppm) | % | (ppm) | % |
| 0 | Control | 43163 | | 6001 | |
| | Effector | 36492 | | 4634 | |
| | Composition | | | | |
| 7 | Control | 39249 | 9.0 | 5067 | 15.5 |
| | Effector | 5390 | 85.2 | 4114 | 11.0 |
| | Composition | | | | |
| 28 | Control | 33961 | 21.0 | 3812 | 36 |
| | Effector | 64 | 99.8 | 1324 | 71 |
| | Composition | | | | |

TABLE 2
Product Comparison Chart.

| | | | Alkanes | | Aromatics | |
|---|---|---|---|---|---|---|
| Products | Formula | Classification | Day 7 | Day 28 | Day 7 | Day 28 |
| BET | Powder | Biological | — | 99% | — | 67% |
| Biopetro | | | | | | |
| BioWorld | 2-Part* | Biological | 28% | 97% | 40% | 88% |
| BHTP | | | | | | |
| JE1058BS | Powder | Nutrient | — | 92.60% | — | 39% |
| Land/Sea | Powder | Nutrient | 29.50% | 42.92% | — | 31.92% |
| Restoration | | | | | | |
| Micro-Blaze | Liquid | Biological | 56.40% | 94.10% | 54.10% | 47.60% |
| Oil Spill Eater II | Liquid | Enzyme | 36.90% | 89.80% | 33.60% | 89.60% |
| Oppenheimer | Powder | Biological | — | 89.10% | — | 38.20% |
| S-200 | Liquid | Nutrient | — | 98% | — | 10.40% |
| SpillRemed | Liquid | Biological | — | 97% | — | 47% |
| Marine | | | | | | |
| Step One | 2-Part* | Biological | — | 44.03% | — | 54.51% |
| System E.T. | Powder | Biological | — | 99.10% | — | 77% |
| 20 | | | | | | |
| WMI-2000 | Powder | Biological | — | 60.30% | — | 33.30% |
| VB591, | Powder | Nutrient | 49.20% | 96.80% | 21.40% | 73.10% |
| VB997 | | | | | | |
| Soil RX | Liquid | Biological | 30.14% | 49.73% | 46.96% | 73.63% |
| MUNOX SR | 2-Part* | Biological | — | 94.90% | — | 75.50% |
| Effector Composition | Liquid | Biological | 85.20% | 99.80% | 11% | 71% |

Certain modifications and improvements will occur to those skilled in the art upon a reading of the foregoing description. The above-mentioned examples are provided to serve the purpose of clarifying the aspects of the invention and it will be apparent to one skilled in the art that they do not serve to limit the scope of the invention. All modifications and improvements have been deleted herein for the sake of conciseness and readability but are properly within the scope of the present invention.

What is claimed is:

1. A method for effecting an environmental bioremediation comprising the steps of:
providing an effector composition including lignocellulosic particles with at least one absorbed microorganism species therein.

2. A method for effecting an environmental bioremediation comprising the steps of:
providing an effector composition including lignocellulosic particles;
mixing at least one microorganism species with the lignocellulosic particles to form an effector composition.

3. The method of claim 2, further including the step of absorbing the at least one microorganism species into internal spaces of the lignocellulosic particles.

4. The method of claim 3, wherein the lignocellulosic particles are kenaf core particles.

5. The method of claim 2, further including the step of dispersing the effector composition in a target environment for effecting bioremediation thereof.

6. The method of claim 2, wherein the lignocellulosic particles consist substantially of kenaf particles.

7. The method of claim 2, wherein the lignocellulosic particles include kenaf particles.

8. A system for effecting an environmental bioremediation comprising: an effector composition including lignocellulosic particles with at least one absorbed microorganism species therein.

9. The system of claim 8, wherein the lignocellulosic particles include kenaf particles.

10. The system of claim 8, wherein the lignocellulosic particles consist substantially of kenaf particles.
